(19) United States
(12) Patent Application Publication
(10) Pub. No.: US 2011/0313508 A1
(43) Pub. Date: Dec. 22, 2011

(54) ENDOVASCULAR PLATFORMS FOR UNIFORM THERAPEUTIC DELIVERY TO LOCAL TARGETS

(76) Inventors: Vijaya Kolachalama, Cambridge, MA (US); Evan Levine, Auburndale, MA (US); Elazer Edelman, Brookline, MA (US)

(21) Appl. No.: 12/819,429

(22) Filed: Jun. 21, 2010

(51) Int. Cl. A61F 2/82; B23P 11/00

(52) U.S. Cl. 623/1.15; 29/428

(57) ABSTRACT

An endovascular platform is provided that includes a periodic tiling-based stent structure having a parallelogram tile associated with a fundamental domain and an intrinsic cell-shape. A lattice vector includes a section of the parallelogram tile where the lattice vector is circumferentially folded to create helical repetition of cells.

### BACKGROUND OF THE INVENTION

The invention is related to the field of cardiovascular devices, and in particular to an endovascular platform such as a stent that is structurally robust and provides controlled and uniform delivery of therapeutic agents to local targets.

Endovascular platforms such as stents are now routinely used to treat arterial obstructive disease. These metal mesh tubes are mounted in a collapsed or crimped state on a balloon catheter. The catheter is advanced through an artery and across a severely diseased segment. The balloon is inflated, expanding the stent and displacing the arterial obstruction. Without the stent, the balloon inflated artery would recoil and collapse close to its initial obstructed diameter. The presence of the stent prevents this elastic recoil and as it is left expanded in the artery ensures that the artery remains open by virtue of constant outward expansive pressure forces. At the same time, these forces generate obligate equal and opposite set of forces from the artery on the stent. These forces combined with the presence of a permanently indwelling foreign metal stent establish a reactive healing response that eventually causes vascular tissue hyperplasia and growth into and encroaching upon the arterial lumen. This response, termed restenosis, is likely mediated in major part by an attempt to balance wall stress, and the increase in vessel thickness is a result of expanded radius and increased mural pressure. Mechanical and fluid dynamic forces combine with cellular and molecular events to generate this tissue response.

To circumvent the clinical manifestation of restenosis, drugs and other potentially therapeutic agents are coated onto stents and eluted off over time to provide the healing artery with a supply of regulatory compound that minimizes the hyperplastic responses. Since their introduction in 2003, drug-eluting stents (DES) have become the primary choice for the treatment of coronary artery disease. However, recent studies reporting the efficacy of DES have raised questions concerning the longevity of these devices as stent thrombosis emerged as a new fatal complication that presented as myocardial infarction and/or death in some patients.

Vascular lesions treated with DES have delayed and non-uniform endothelialization in comparison to their bare metal counterparts and the time course of tissue healing response is believed to be partially dependent on the transient drug pharmacokinetic properties. Drugs inhibit vascular neointimal hyperplastic response as well as delay the process of healing characterized by the formation of endothelial lining over the stented region. Further, drug deposition patterns established due to local delivery from discrete mesh-like structures such as stents inherently create regions of high concentrations of drug that induce vascular toxicity and zones of low concentrations of drug that can cause local re-narrowing. There is a growing body of evidence that this spatial heterogeneity in drug deposition is caused due to coupling of convective and diffusive transport forces and that luminal blood flow plays a major role in determining the arterial drug distribution. These non-uniform drug distribution patterns prevail for all the current commercial stent designs and therefore, there is an urgent need to develop better designs or optimize existing ones to minimize the discrepancy in arterial drug distribution patterns which in turn could reduce the potential risk of stent thrombosis as well as inhibit restenosis.

### SUMMARY OF THE INVENTION

According to one aspect of the invention, there is provided an endovascular stent structure. The endovascular stent structure includes a periodic tiling-based stent structure having a parallelogram tile associated with a fundamental domain and an intrinsic cell-shape. A lattice vector includes a section of the parallelogram tile where the lattice vector is circumferentially folded to create helical repetition of cells.

According to another aspect of the invention, there is provided a method for designing an endovascular stent structure. The method includes providing a periodic tiling-based stent structure having a parallelogram tile associated with a fundamental domain and an intrinsic cell-shape. The method includes forming a lattice vector having a section of the parallelogram tile. Also, the method includes folding the lattice vector to create helical repetition of cells.

### BRIEF DESCRIPTION OF THE DRAWINGS

FIG. 1 is a schematic diagram illustrating a number of periodic stent designs with their respective fundamental domain (FD) and intrinsic cell shapes 18;

FIG. 2 is a schematic diagram illustrating the process flow for designing stents in accordance with the invention;

FIG. 3 is a schematic diagram illustrating a lattice vector formed in accordance with the invention;

FIG. 4 is a table illustrating mathematical equations used in accordance with the invention;

FIG. 5 is a schematic diagram illustrating a parallelogram cell for regular hexagonal tiling;

FIGS. 6A-6C are schematic diagrams illustrating several configurations with varying lattice vectors for a hexagonal shape cell-based stent formed in accordance with the invention;

FIGS. 7A-7C are schematic diagrams illustrating a known stent structure and its respective fundamental domain and intrinsic cell shape and how changes in the lattice vectors change the stent structure; and

FIG. 8A-8B are schematic diagrams illustrating the drug distribution of a known stent structure and that of a stent structure changed in accordance of the invention.

### DETAILED DESCRIPTION OF THE INVENTION

The invention is based on the appreciation that drug release and subsequent arterial distribution is dependent on the endovascular platform's design characteristics and is mediated by principles of convection and diffusion. Stent-based delivery leads to spatially heterogeneous drug concentration patterns and minimization of this non-uniformity can lead to more desirable clinical outcomes. The underlying premise is that high drug concentration gradients within the lesion site are not clinically favorable and can lead to non-uniform healing response because the drugs used for inhibiting neointimal hyperplastic response have a narrow therapeutic window. Specifically, high drug concentration regions are toxic and could be thrombogenic whereas low drug concentration regions have no effect in inhibiting restenosis where the associated sites may cause luminal re-narrowing. The invention proposes a method for modifying any existing stent design such that homogeneity in the arterial drug distribution pattern is maximal.

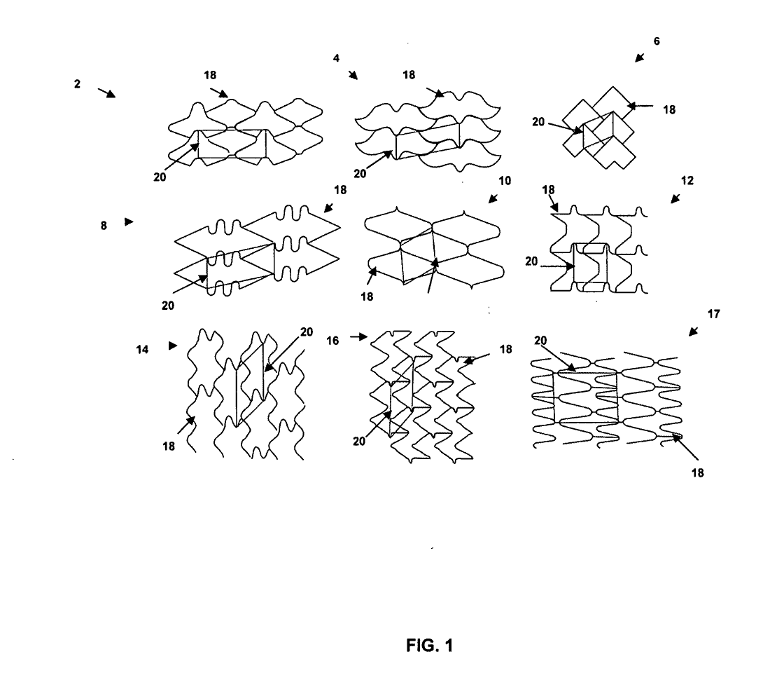

Endovascular stent designs have been categorized as either slotted-tube or corrugated ring, and either multi-link or open cell. For example, the US Food and Drug Administration approved DES such as Cypher is a slotted-tube design whereas the recently approved DES such as Xience is a multi-link design. FIG. 1 shows a number of stents 2-17 that can be used in accordance with, the invention. All these stents 2-17 are mesh-like cylindrical domains and each design is characterized by a unique intrinsic structure, denoted as a cell 18 in FIG. 1. Conventional paradigm identifies a single cell for a design as a region enclosed by stent struts. These cells 18 have no consistent shape and are dependent on the specific stent geometry. It is intuitive to imagine that a stent with given radial and axial dimensions is generated when a series of these cells 18 are tiled both in the circumferential and longitudinal directions.

One can use tiling principles to provide a generalized intrinsic element for any repetitive mesh-like cylindrical construct such as an endovascular stent. The tiling of the plane is defined as an arrangement of disjoint shapes which leaves no gaps. Periodic tilings are translation invariant along two independent directions, and non-periodic tilings are not. All mesh-like devices have patterns that appear the same along their length and circumference, and thus may be derived from periodic tilings. A generalized intrinsic element arises by defining periodic tilings and their cellular shapes by a parallelogram-shaped region, henceforth denoted as a fundamental domain (FD) 20 as shown in FIG. 1. An FD 20 does not generally have boundaries along stent struts, but contains stent segments. When tiled in the plane, the FD 20 generates the endovascular platform's cellular pattern as adjacent FD's form strut interconnections. A typical FD can be identified in any periodic tiling, and therefore, the invention is applicable to all endovascular platforms including stents and stent-grafts based on periodic things, as show in FIG. 1. Each FD 20 has a particular shape and orientation relative to each endovascular platform pattern. Fundamental domains are unique for any given endovascular platform in the sense that they have only one possible size, shape, and orientation which are determined by the underlying periodic cell tiling.

Fundamental domains are not unique. For any valid FD, alternative FD's can be identified with identical shapes and orientations with respect to the underlying pattern. However, the parallelogram with the appropriate shape and orientation can be positioned anywhere over the tiling to represent an intrinsic cell shape, and therefore the FD's position is chosen as an arbitrary reference. Fully defined by this position, the FD can be “copied” and tiled periodically to form an infinite arrangement of identical elements, denoted as a lattice.

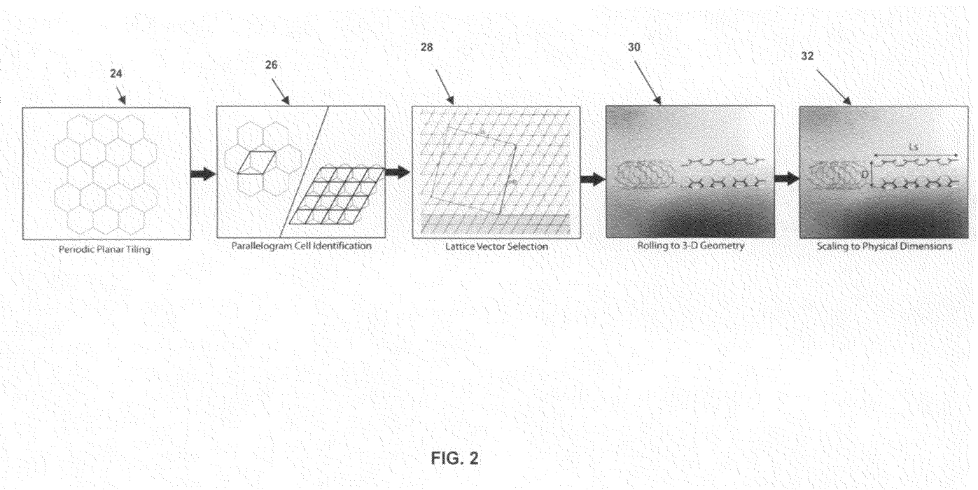

The invention uses the lattice to develop novel endovascular platforms such as stents with cell periodicities that are skewed with respect to longitudinal and circumferential directions. Tiled periodically in the plane, FD's are arranged in a lattice of identical elements which together map out the strut pattern for the periodic endovascular platform design. The lattice alone only partially defines the endovascular platform's layout in three-dimensions. To map from the lattice to a three-dimensional design, the subsequent step is to “cut” a rectangular region from the lattice containing many FD's and roll it into a cylinder, which can be scaled to the desired dimensions, as shown in FIG. 2.

In particular, FIG. 2 shows the process flow of the sequential design methodology. The methodology begins with any periodic tiling-based endovascular platform like a stent, as shown in step 24, and identifies the corresponding parallelogram tiling associated with a FD of the periodic tiling-based endovascular platform, as shown in step 26. Later, a lattice vector incorporating a section of the parallelogram tile is extracted as shown in step 28 and circumferentially folded to create a three dimensional endovascular platform as shown in step 30. Finally, the resulting platform can be uniformly scaled to desirable physical dimensions as shown in step 32.

There are many lattice vectors that can be used in step 28 to generate helical endovascular platform designs, and each lattice vector introduces a different degree of helicity visible in the three-dimensional cell pattern. Lattice vectors within the span of two periodic directions of the periodic tiling generate conventional non-helical endovascular platforms, and all other lattice vectors generate stents with helical shapes. The invention includes all possible three-dimensional embodiments of the lattice with cells repeating in a helical fashion.

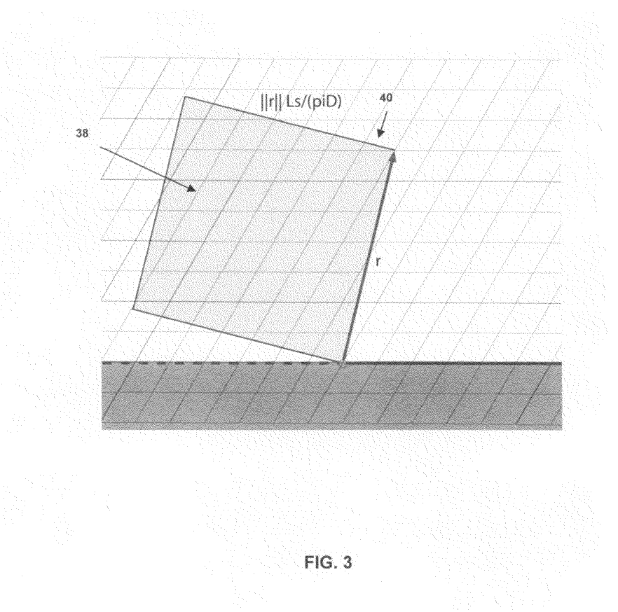

The invention uses five parameters to describe any three-dimensional endovascular platform: a definition of the FD, a diameter (D), length (Lₛ), strut thickness (T), and lattice vector ( [symbol] ). The planar periodic tiling for a design can be transferred to a cylindrical surface via the latter parameter, which has endpoints at distinct vertices of the lattice and prescribes a fold used to create the endovascular platform such as a stent. The lattice vector and an orthogonal vector form a rectangular region with proportions πD: Lₛ, as shown in FIG. 3.

Extracted from the lattice, the rectangular region 30 alone can be rolled into an endovascular platform design with a continuous pattern over the entire surface. FD's are split at the boundaries of the rectangular region, but join with complementary FD's at the opposite side. Finally, the parameters T, D, and Lₛ scale the design to physical dimensions.

To enumerate the lattice vectors which generate unique designs, the lattice vector [symbol] is denoted in a coordinate system and a condition is imposed to account for an inherent symmetry of the stent. First, an x-y coordinate system is defined with origin at an arbitrary vertex of any parallelogram and x-axis collinear with either side of the parallelogram 40, as shown in FIG. 3. The x-axis scale is set by placing the first vertex on x>0 at x=1. One can define two unit vectors, \vec{u} and \vec{v} with coordinates of the parallelogram vertices adjacent to the vertex at the origin, with \vec{u}=(1,0). The lattice vector is then defined as nu+mv and denoted [symbol] =<n,m>, where n and m are integers. This lattice vector matches vertices of the lattice which generate an endovascular platform design. However, <n,m> and <−n,−m> represent equivalent designs, and consequently, it is sufficient to consider only the set of lattice vectors for which m≧0 for n>0 and m>0 for n≦0. Furthermore, it may be necessary to account for other possible symmetries of the tiling for which the set of lattice vectors generate two or more equivalent designs.

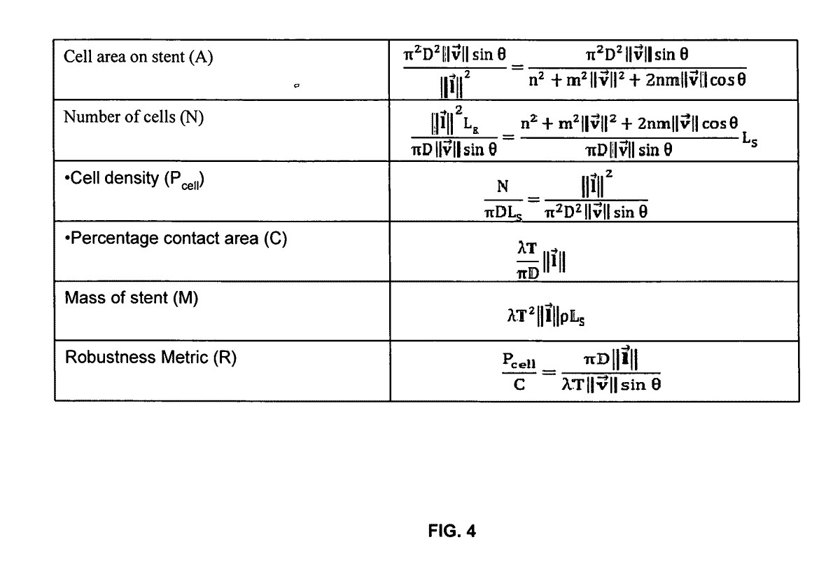

The five aforementioned independent parameters alter several characteristics of the design, including the FD area, degree of helicity of the endovascular platform design, the number of cells, mass of the stent, and contact between endovascular platform material and tissue. The lattice vector and fundamental domain together define a three-dimensional geometric pattern, and Lₛ, T, and D then provide a physical scale. The FD's proportions are given by an angle θ between \vec{u} and \vec{v} and |\vec{v}|, which are fixed after it is defined, but its physical area is dependent on [symbol] , D, and the FD's proportions. The angle of the lattice vector, φ, measured from the positive x-axis, can be used to measure the helicity of the cellular pattern on the stent by comparing φ to θ. The design obtained from [symbol] generally appears most helical when for φ=θ/2, and non-helical when φ=0 or φ=θ. A metric for helicity (h) can be defined as |φ−2|. Designs for which h=0 have no helical repetition of cellular elements and thus appear in the form of conventional designs. Their cells are periodic in longitudinal and circumferential directions and form closed rings over their circumferences. The number of cells is denoted by N and the cell density (P_{cell}) is defined as the number of cells per unit area of the stent. The mass of the stent is defined as M. The contact between the stent material and tissue is best interpreted as a percent coverage of the endovascular platform, defined as C, the ratio of the contact area between the endovascular platform and tissue to the total area of tissue in the deployed region of the vessel. The ratio is dependent on the strut thickness (T) and the average arc-length of strut curves per unit of cell area (λ). FIG. 4 shows a table of the mathematical relationships for cell area on stent (A), number of cells (N), cell density (P_{cell}), percentage contact area (C), mass of stent (M), and robustness metric (R).

The methodology described and equations shown in FIG. 4 can be used to achieve another dimension of control of the stent-tissue system's fluid mechanical and solid mechanical characteristics. Each direction of cell periodicity can affect luminal flow patterns both proximal and distal to the endovascular platform as well as within the deployed region. This mural drug deposition pattern ultimately governs the amount of drug uptake in the tissue beneath and the resulting efficacy of the intervention. By modifying the angle φ of the lattice vector, it is possible to construct cellular patterns with any degree of helicity. Accordingly, the lattice vector angle φ is expected to serve as the principal control parameter for flow. The lattice vector can also be modulated with regard to solid mechanical aspects.

The cell density P_{cell} increases the radial support provided by the endovascular platform as each cell provides a point of support. P_{cell} increases in proportion to the square magnitude of the lattice vector, so longer lattice vectors provide more support by generating densely packed cells. Simultaneously, the percent contact area C increases with P_{cell}. To represent the contribution of material to the formation of cells, one can define a robustness metric R as P_{cell}/C and attempt to optimize the design with respect to R.

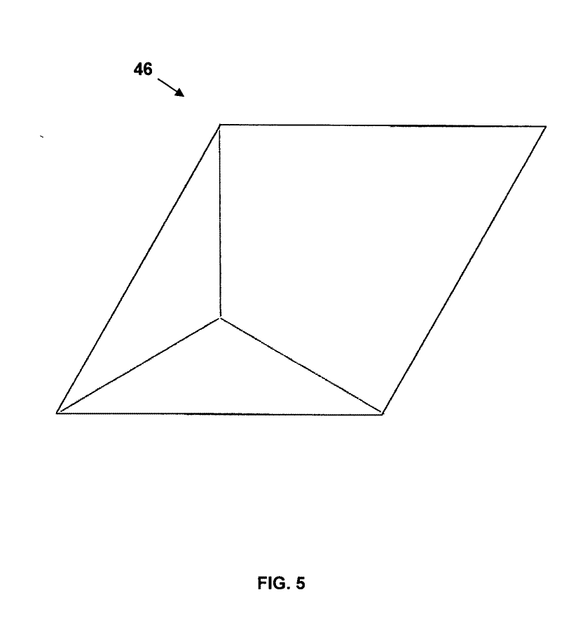

In another aspect of the invention, optimizing R of a cell shape leads to generation of a regular hexagonal (honeycomb) tiling. FIG. 5 shows a parallelogram cell 46 for regular hexagonal tiling. The regular hexagon can be embedded within a parallelogram cell 46, which generates a regular hexagonal tiling. The hexagonal tiling has unique mathematical properties and has been observed in nature to be a robust structure. It appears on the strongest engineering material, the carbon nanotube, a cylindrical allotrope of carbon arranged in a hexagonal tiling. The proof of the “honeycomb conjecture” shows that the regular hexagonal tiling uses the least total perimeter to tile the plane of any cell shape with fixed area and helps to explain the commonality of the hexagonal patterning in nature and its optimality in physical and biological structures. The unique perimeter-minimizing property of the hexagon is also proven for patterns over cylindrical surfaces. It follows from the proof that the hexagon minimizes λ and therefore the percent contact area C for a fixed [symbol] , D, |v|sin θ, and T. Its associated FD then maximizes R.

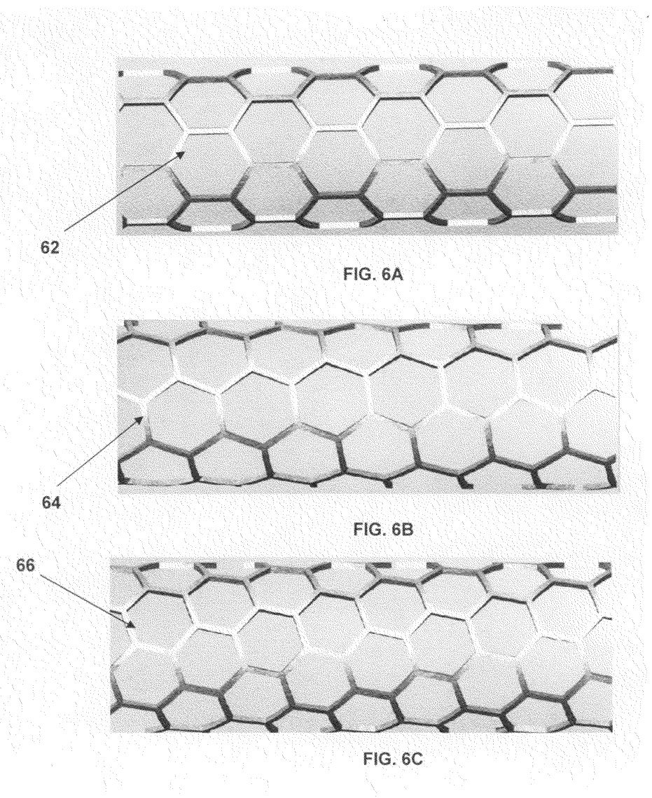

Moreover, one can show that it is only necessary to consider lattice vectors with angles 0° to 30° because the associated lattice has 30° rotational symmetry. 120 possible designs were considered. Models 62, 64, 66 with lattice vectors <7, 0>, <4, 4>, and <5, 3> and respective cell areas 1.57 mm², 1.60 mm², and 1.57 mm² were chosen, as shown in FIGS. 6A-6C.

Similar to the example mentioned above, the invention can be easily implemented for other types of designs. In this way, the overall structural properties of the endovascular platform are not significantly altered but the variation in flow-mediated drug distribution pattern within the deployed site can be significant.

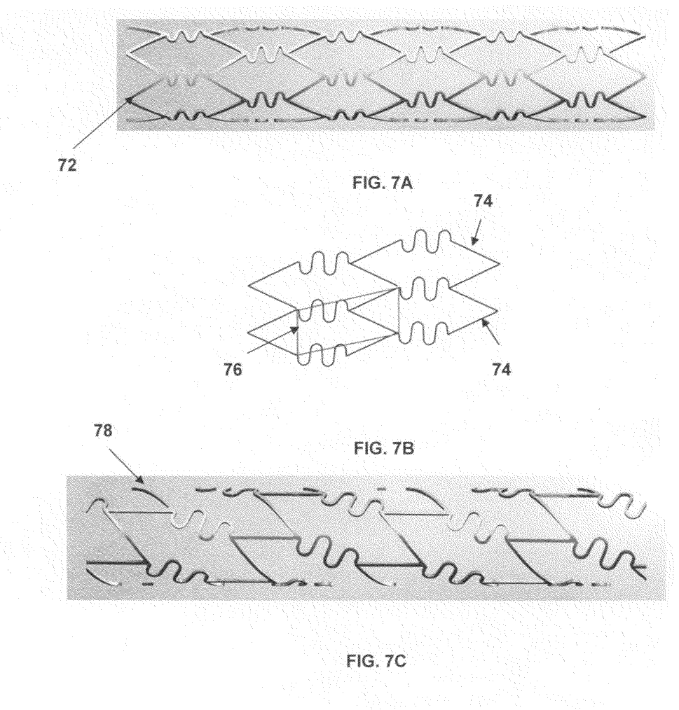

Design of slotted-tube stents such as Cypher can be easily modified by first identifying the fundamental domain and subsequently altering the lattice vector. FIG. 6A shows the conventional configuration of the Cypher stent available for clinical use. FIG. 7B shows the fundamental domain 76 identified using the invention and the shape of an intrinsic cell 74. FIG. 7C shows a new stent configuration 78 being created which maintains a similar cell shape but a change in the orientation of the fundamental domain due to a variation in the lattice vector creates.

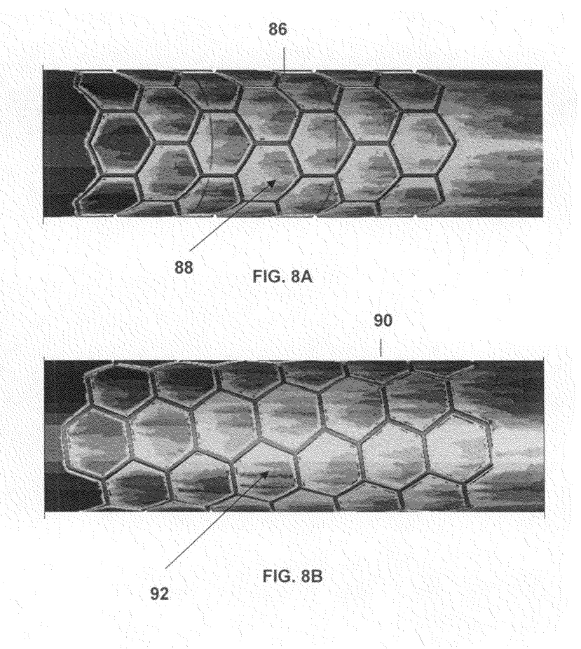

Note drug distribution patterns for stents created with varying lattice vectors can be significantly different. It is possible to choose the optimal lattice vector parameters such that arterial drug distribution patterns within the platform-implanted site are more uniform. Conventional wisdom based traditional stent designs 86 lead to drug distribution patterns 88 that are spatially varying, shown in FIG. 8A, and by optimizing the lattice vector parameters, one can create a stent design 90 where the drug distribution is more homogenous along the entire length of the stent, as shown in FIG. 8B.

The invention has great economic potential as it is applicable for any form of endovascular device regardless of whether they are drug-eluting or non-eluting. For non-eluting devices, the optimal lattice vector parameters correspond to designs that are structurally robust and induce uniform loading conditions on the arterial vessel. For the case of drug-eluting devices, both the structural and pharmacokinetic aspects are optimized. The invention is also applicable to all forms of bio-degradable, bio-absorbable as well as bio-compatible stents or stent-grafts. The paradigm can be extended to any form of either slotted-tube or corrugated ring, and either multi-link or open cell designs. Device manufacturers can achieve a new dimension of control for designing structurally more robust designs by simply varying the aforementioned parameters defining the lattice vector. Additionally, by carefully adjusting the lattice vector parameters, spatial distribution of arterial drug can be maintained at uniform levels to create clinically favorable outcomes.

Although the present invention has been shown and described with respect to several preferred embodiments thereof, various changes, omissions and additions to the form and detail thereof, may be made therein, without departing from the spirit and scope of the invention.

What is claimed is:

1. An endovascular platform structure comprising:
a periodic tiling-based stent structure having a parallelogram tile associated with a fundamental domain and an intrinsic cell-shape; and
a lattice vector having a section of the parallelogram tile where the lattice vector is circumferentially folded to create helical repetition of cells.

2. The endovascular platform of claim 1, wherein the fundamental domain defines a strut pattern for the periodic tiling-based stent structure.

3. The endovascular platform of claim 1, wherein the lattice vector is circumferentially folded to form a cylinder.

4. The endovascular stent structure of claim 1, wherein the cylinder is scaled to dimensions.

5. The endovascular platform of claim 1, wherein the endovascular platform comprises a three dimensional shape.

6. The endovascular platform of claim 1, wherein the lattice vector is varied to adjust drug distribution patterns.

7. The endovascular platform of claim 6, wherein the distribution patterns comprise a homogenous profile.

8. The endovascular platform of claim 6, wherein the intrinsic cell-shape is maintained.

9. The endovascular platform of claim 1, wherein the parallelogram tile comprises a plurality of the fundamental domains.

10. A method for designing an endovascular platform comprising:
providing a periodic tiling-based stent structure having a parallelogram tile associated with a fundamental domain and an intrinsic cell-shape;
providing a lattice vector having a section of the parallelogram tile; and
folding the lattice vector to create helical repetition of cells.

11. The method of claim 10, wherein the fundamental domain defines a strut pattern for the periodic tiling-based stent structure.

12. The method of claim 10, wherein the lattice vector is circumferentially folded to form a cylinder.

13. The method of claim 10, wherein the cylinder is scaled to dimensions.

14. The method of claim 10, wherein the endovascular platform comprises a three dimensional shape.

15. The method of claim 10, wherein the lattice vector is varied to adjust drug distribution patterns.

16. The method of claim 15, wherein the distribution patterns comprise a homogenous profile.

17. The method of claim 15, wherein the intrinsic cell-shape is maintained.

18. The method of claim 10, wherein the parallelogram tile comprises a plurality of the fundamental domains.
